(19) United States
(12) Patent Application Publication
(10) Pub. No.: US 2003/0099711 A1
(43) Pub. Date: May 29, 2003

(54) Sustained release preparations

(76) Inventors: David Meadows, Colleyville, TX (US); Peter Young, Westover Hills, TX (US); Donald J. Keyser, South Lakes, TX (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 10/230,263

(22) Filed: Aug. 29, 2002

Related U.S. Application Data
(60) Provisional application No. 60/315,328, filed on Aug. 29, 2001.

(51) Int. Cl. A61L 2/00; B05D 3/00; A61K 9/22; A61K 9/28

(52) U.S. Cl. 424/474; 427/2.1; 604/500; 424/468

(57) ABSTRACT

This invention relates to oral pharmaceutical preparations that comprise a pharmacologically active drug bound to small particles of an ion-exchange resin. Drug-resin complexes are coated with an aqueous based diffusion barrier comprising a water-permeable, film forming polymer that is relatively insoluble in gastrointestinal fluids thereby providing a controllable sustained release of drug under conditions encountered in the gastrointestinal tract. At least some of the barrier coated drug-resin particles may be coated with an enteric coating to provide a tailored release profile.

### FIELD OF THE INVENTION

The invention is directed to oral preparations comprising at least one pharmacologically active drug bound to small particles of an ion-exchange resin to provide a drug-resin complex which results in the prolonged release of the drug. Drug-resin complexes can be coated with a water-permeable diffusion barrier coating that is insoluble in gastrointestinal fluids thereby providing a controllable sustained release of drug under conditions encountered in the gastrointestinal tract. A second coating of the drug resin complex particles may be provided with an enteric coating to formulate tailored release profiles. The preferred formulation is a liquid suspension of the coated drug/ion-exchanger resin complex.

### BACKGROUND OF THE INVENTION

Sustained or prolonged-release dosage forms provide a controlled and constant supply of drug to an organism. Controlled release drugs preparations provide the convenience of daytime dosing where the dosage form can be taken first thing in the morning and provide therapeutic levels of the drug throughout the day. Further, a controlled-release drug preparation delivers drugs in a manner that will maintain therapeutically effective plasma levels over a period of time that is significantly longer than that which is given by a typical drug dosage form. This eliminates the need to interrupt sleep to take medication and can prevent missed doses, thus improving patient compliance. Benefits obtained from such a controlled release of a specific drug include the control of cough, sleep, enuresis, pain and migraine headaches. Additionally, controlled release of antimicrobials can be obtained to treat or prevent infection.

Uncoated ion-exchange resin-drug complexes which delay release of a drug in the gastrointestinal tract are described in U.S. Pat. No. 2,990,332. However, uncoated complexes provide only a relatively short delay of drug release and a poor control of drug release because the control is limited to variation in particle size and cross-linkage of the sulfonic acid-type resin used to prepare the adsorption compounds. Various coated resin-drug complexes have been reported (e.g., U.S. Pat. Nos. 3,138,525; 3,499,960 and 3,594,470; Belgian Patent No. 729,827; German Patent No. 2,246,037; and Borodkins et al., Journal of Pharmaceutical Science, Vol. 60, pages 1523-1527, 1971).

Water-permeable diffusion barrier coated drug/resin complexes can undergo significant swelling (up to about a 60% increase in volume) when the dry, non-hydrated form is placed in contact with gastrointestinal fluids. This swelling can rupture the diffusion barrier coating and result in loss of control of the diffusion of released drug.

Controlled-release drugs for use in the gastrointestinal tract are described in U.S. Pat. No. 4,221,778. The method described therein for preparing products having controlled release properties involved a three-step process: (i) preparation of a drug-resin complex; (ii) treating this complex with a suitable impregnating agent; and (iii) coating the particles of treated complex with a water-permeable diffusion barrier. The use of impregnation agents is believed to prevent swelling or rupturing of the barrier coating. This patent is hereby incorporated by reference.

Other patents that describe improvements and variations of this type of product include U.S. Pat. Nos. 4,996,047; 5,186,930; 4,894,239; 4,859,462; 4,959,219; 4,847,007; 4,762,709; 4,999,189; 4,859,461; and 5,368,852, all of which are hereby incorporated by reference.

The use of enteric coatings to delay drug release until the product leaves the stomach are also known. See for example U.S. Pat. No. 5,851,579, which is hereby incorporated by reference.

### SUMMARY OF THE INVENTION

The present invention overcomes the problems and disadvantages associated with current strategies and designs and provides products and methods for the controlled-release of drug compositions.

One embodiment of the invention encompasses particles that comprise a drug complexed with a pharmaceutically acceptable ion-exchange resin. The resulting drug-resin particles can be coated with a substance that acts as a barrier to control the diffusion of the drug into gastrointestinal fluids.

Another embodiment of the invention encompasses drug-resin particles coated with an enteric coating. Yet another embodiment of the invention encompasses drug-resin particles coated with a first coating, a diffusion barrier coating, and a second coating, an enteric coating.

Another embodiment of the invention encompasses pharmaceutical compositions comprising at least two of particles selected from drug-resin particles, drug diffusion coated drug-resin particles, enteric coated drug-resin particles, and drug diffusion and enteric coated drug-resin particles. Yet another embodiment of the invention encompasses pharmaceutical compositions comprising at least two drug-resin particles having different delayed release coatings, i.e., mixtures of drug-resin particles having different amounts of drug-barrier coating. Tailored release profile pharmaceutical formulations can be made with mixtures of at least two of the particles described above.

Another embodiment of the invention is directed to methods for the manufacture of particles described above.

Another embodiment of the invention is directed to methods for the controlled release of at least one drug.

Other embodiments and advantages of the invention are set forth in part in the description which follows, and in part, will be obvious from this description, or may be learned from the practice of the invention.

### BRIEF DESCRIPTION OF THE DRAWINGS

FIG. 1 is a graph showing a serum profile of concentration versus time for a controlled release composition according to the invention.

FIG. 2 is graph showing a serum profile of concentration versus time for another controlled release composition according to the invention.

FIG. 3 is a graph showing a serum profile of concentration versus time for another controlled release composition according to the invention.

FIG. 4 illustrates the percent PPA released of untreated drug-resin particles, water-soluble barrier coated drug-resin, and barrier coated drug-resin formulations of the invention at initial time zero and after a two hour period.

FIG. 5 illustrates the percent dextromethorphan released of untreated drug-resin particles, five barrier coated drug-resin formulations of the invention, and commercially available Delsym™ over a two hour period.

FIG. 6 illustrates a dissolution study of dextromethorphan using formulations of the present invention as compared to commercially available Delsym™ over a 12 hour period.

### DETAILED DESCRIPTION OF THE INVENTION

As embodied and described herein, the present invention is directed to delayed release drug formulations comprising drug-resin complexes that can be used for the prolonged in vivo release of pharmaceutical preparations. Optionally, the drug-resin complexes may have at least one coating, wherein the coating may be of different weight diffusion release coatings, an enteric coating, or combinations thereof. Also, the invention is directed to methods for the manufacture of the drug-resin particles and their use for the controlled, in vivo release of pharmaceutically active drugs.

The treatment, control, and amelioration of disorders and/or the control of symptoms are basic goals of drug therapy. One aspect of all drug therapy is the sustained administration of an effective dose of drug for an extended period of time. In many cases, the longer the period of time, the more substantial the benefit. Sustained or prolonged-release dosage forms of various drugs are known and commercially available. In one method, drug is complexed with resin forming a particle. After administration, the drug is slowly released from the resin over time thereby providing constant or near constant delivery of drug to the patient. These particles, however, are difficult and expensive to manufacture requiring multiple steps and a coating which must first be dissolved in a non-aqueous solvent, some of which remains in the final product. It has been surprisingly discovered, that controlled-release particles containing pharmaceutically active drug can be manufactured using aqueous materials for the coating. Although such coatings are sufficiently larger and thicker than would be expected by one of ordinary skill in the art, as such, particle manufacture is still simpler, less expensive, and requires no non-aqueous solvent during manufacture or processing resulting in a cleaner, safer product.

Accordingly, one embodiment of the invention is directed to drug-resin particles that provide a controlled supply of drug to an organism. The controlled release aspect is achieved by complexing drug to resin forming drug-resin particles, and application to the particles of a diffusion barrier comprising a water-permeable, film-forming polymer, an enteric coating, or both. The use and advantages of employing aqueous dispersions of the barrier polymer are disclosed. Upon administration to a patient, fully coated solvent-free drug-resin particles provide a controlled release of at least one active drug. Drug-resin particles of the invention are briefly described as follows:

Resin

Ion-exchange resins suitable for use in these preparations are water-insoluble and comprise a pharmacologically inert organic and/or inorganic matrix containing covalently bound functional groups that are ionic or capable of being ionized under the appropriate conditions of pH. The organic matrix may be synthetic (e.g. polymers or copolymers of acrylic acid, methacrylic acid, sulfonated styrene, sulfonated divinylbenzene), or partially synthetic (e.g. modified cellulose and dextrans). The inorganic matrix preferably comprises silica gel modified by the addition of ionic groups. Covalently bound ionic groups may be strongly acidic (e.g., sulfonic acid, phosphoric acid), weakly acidic (e.g., carboxylic acid), strongly basic (e.g., primary amine), weakly basic (e.g. quaternary ammonium), or a combination of acidic and basic groups. In general, the types of ion-exchangers suitable for use in ion-exchange chromatography and for such applications as deionization of water are suitable for use in the controlled release of drug preparations. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975). Ion-exchange resins that can be used in the present invention have exchange capacities below about 6 milliequivalents (meq)/gram and preferably below about 5.5 meq/gram.

Typically, the size of the ion-exchange particles is from about 30 microns to about 500 microns, preferably the particle size is within the range of about 40 micron to about 150 micron for liquid dosage forms although particles up to about 1,000 micron can be used for solid dosage forms, e.g., tablets and capsules. Particle sizes substantially below the lower limit are difficult to handle in all steps of the processing. Commercially-available ion-exchange resins having a spherical shape and diameters up to about 1,000 micron, are gritty in liquid dosage forms and have a greater tendency to fracture when subjected to drying-hydrating cycles. Moreover, it is believed that the increased distance that a displacing ion must travel in its diffusion into these large particles, and the increased distance the displaced drug must travel in its diffusion out of these large particles, cause a measurable but not readily controlled prolongation of release even when the drug-resin complexes are uncoated. Release of drug from uncoated drug-resin complexes with particle sizes in the approximate range of 40 micron to 150 micron is relatively rapid. Satisfactory control of the release from such complexes is achieved almost exclusively by the applied diffusion barrier coating.

Both regularly and irregularly shaped particles may be used as resins. Regularly shaped particles are those particles that substantially conform to geometric shapes such as spherical, elliptical, cylindrical and the like, which are exemplified by Dow XYS-40010.00 and Dow XYS-40013.00 (The Dow Chemical Company). Irregularly shaped particles are all particles not considered to be regularly shaped, such as particles with amorphous shapes and particles with increased surface areas due to surface channels or distortions. Irregularly shaped ion-exchange resins of this type are exemplified by Amberlite IRP-69 (Rohm and Haas). Two of the preferred resins of this invention are Amberlite IRP-69 and Dow XYS-40010.00. Both are sulfonated polymers composed of polystyrene cross-linked with 8% of divinylbenzene, with an ion-exchange capacity of about 4.5 to 5.5 meq/g of dry resin (H⁺-form). Their essential difference is in physical form. Amberlite IRP-69 consists of irregularly-shaped particles with a size range of 47 micron to 149 micron produced by milling the parent large-sized spheres of Amberlite IRP-120. The Dow XYS-40010.00 product consists of spherical particles with a size range of 45 micron to 150 micron. Another useful exchange resin, Dow XYS-40013.00, is a polymer composed of polystyrene cross-linked with 8% of divinylbenzene and functionalized with a quaternary ammonium group; its exchange capacity is normally within the range of approximately 3 to 4 meq/g of dry resin.

Drugs

Drugs that are suitable for use in these preparations include drugs for the treatment of respiratory tract disorders such as, for example, antitussive expectorants such as dihydrocodeine phosphate, codeine phosphate, noscapine hydrochloride, phenylpropanolamine hydrochloride, potassium guaiacolsulfonate, cloperastine fendizoate, dextromethorphan hydrobromide and chloperastine hydrochloride; bronchodilators such as dl-methylephedrine hydrochloride and dl-methylephedrine saccharinate; and antihistamines such as fexofenadine HCl or dl-chlorpheniramine maleate. Other drugs useful for the invention include drugs for the treatment of digestive tract disorders such as, for example, digestive tract antispasmodics including scopolamine hydrobromide, metixene hydrochloride and dicyclomine hydrochloride, drugs for the treatment of central nervous system disorders such as, for example, antipsychotic drugs including phenothiazine derivatives (chlorpromazine hydrochloride, etc.) and phenothiazine-like compounds (chlorprothixene hydrochloride, etc.), antianxiety drugs such as benzodiazepine derivatives (chlordiazepoxide hydrochloride, diazepam, etc.), antidepressants such as imipramine compounds (imipramine hydrochloride, etc.), antipyretic analgesics such as sodium salicylate, and hypnotics such as phenobarbital sodium; opioid analgesic drugs such as alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethotheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavretum, pentazocine, phenadoxone, phenomorphan, phenazocine, phenoperidine, piminodine, piritramide, propheptazine, promedol, properidine, propiram, propoxyphene, sufentanil, tramadol, tilidine, salts thereof, mixtures of any of the foregoing, mixed mu-agonists/antagonists, mu-antagonist combinations, and the like; and drugs for the treatment of respiratory system disorders such as, for example, coronary dilators including etafenone hydrochloride, antiarrhythmics such as procainamide hydrochloride, calcium antagonists such as verapamil hydrochloride, hypotensive drugs such as hydrazine hydrochloride, propranolol hydrochloride and clonidine hydrochloride, and peripheral vasodilators/vasoconstrictors such as tolazoline hydrochloride. Antibiotics may also be useful such macrolides such as oleandomycin phosphate, tetracyclines such as tetracycline hydrochloride, streptomycins such as fradiomycin sulfate, and penicillin drugs such as dicloxacillin sodium, pivmecillinam hydrochloride and carbenicillinindanyl sodium. Chemotherapeutic drugs may also be used including sulfa drugs such as sulfisomidine sodium; antituberculosis drugs such as kanamycin sulfate, and antiprotozoan drugs such as amodiaquine hydrochloride. An excellent sustained releasing effect is obtained in basic drugs for the respiratory tract such as dihydrocodeine phosphate, dl-methyl-ephedrine hydrochloride and phenylpropanolamine hydrochloride. Additionally, drugs that are suitable for the invention may be acidic, basic or amphoteric. Acidic drugs that can be used in the present invention include, for example, dehydrocholic acid, diflunisal, ethacrynic acid, fenoprofen, furosemide, gemfibrozil, ibuprofen, naproxen, phenytoin, probenecid, sulindac, theophylline, salicylic acid and acetylsalicylic acid. Basic drugs that can be used in the present invention include, for example, acetophenazine, amitriptyline, amphetamine, benztropine, biperiden, bromodiphenhydramine, brompheniramine, carbinoxamine, chloperastine, chlorcyclizine, chlorpheniramine, chlorphenoxamine, chlorpromazine, clemastine, clomiphene, clonidine, codeine, cyclizine, cyclobenzaprine, cyproheptadine, desipramine, dexbrompheniramine, dexchlorpheniramine, dextroamphetamine, dextromethorphan, dicyclomine, diphemanil, diphenhydramine, doxepin, doxylamine, ergotamine, fluphenazine, haloperidol, hydrocodone, hydroxychloroquine, hydroxyzine, hyoscyamine, imipramine, levopropoxyphene, maprotiline, meclizine, mepenzolate, meperidine, mephentermine, mesoridazine, methadone, methylephedrine, methdilazine, methscopolamine, methysergide, metoprolol, nortriptylene, noscapine, nylindrin, orphenadrine, papaverine, pentazocine, phendimetrazine, phentermine, phenylpropanolamine, pyrilamine, tripelennamine, triprolidine, promazine, propoxyphene, pro panolol, pseudoephedrine, pyrilamine, quinidine, scopolamine, dextromethorphan, chlorpheniramine and codeine. Amphoteric drugs that can be used in the present invention include, for example, aminocaproic acid, aminosalicylic acid, hydromorphone, isoxsuprine, levorphanol, melphalan, morphine, nalidixic acid, and paraaminosalicylic acid.

Other drugs which may be used in the invention include, methylphenidate, dexmethylphenidate, oxymorphone, codeine, hydrocodone, chloropheniramine, niacin, aspirin, salts thereof, and combinations thereof. Salts include, but are not limited to, methylphenidate HCl, dexmethylphenidate HCl, oxymorphone HCl, codeine phosphate, hydrocodone bitartrate, chlorpheniramine polistirex, and salicyates.

Drug-Resin Complexes

Binding of drug to resin can be accomplished using methods known in the art, one of ordinary skill in the art with little or no experimentation can easily determine the appropriate method depending upon the drug. Typically four general reactions are used for a basic drug, these are: (a) resin (Na³⁰-form) plus drug (salt form); (b) resin (Na³⁰-form) plus drug (as free base); (c) resin (H⁺-form) plus drug (salt form); and (d) resin (H⁺-form) plus drug (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d). Without being limited by theory, it is believed that the extent of drug binding is critical to the maintenance of the integrity of the diffusion barrier coating.

Four analogous binding reactions can be carried out for binding an acidic drug to an anion exchange resin. These are: (a) resin (Cl⁻-form) plus drug (salt form); (b) resin (Cl⁻-form) plus drug (as free acid); (c) resin (OH⁻-form) plus drug (salt form); and (d) resin (OH⁻-form) plus drug (as free acid). All of these reactions except (d) have ionic by-products and the anions generated when the reactions occur compete with the anionic drug for binding sites on the resin with the result that reduced levels of drug are bound at equilibrium. For acidic drugs, stoichiometric binding of drug to resin is accomplished only through reaction (d). The binding may be performed, for example, as a batch or column process, as is known in the art. The drug-resin complexes may be prepared by a batch process that is based on reaction (d). The drug-resin complex thus formed is collected by filtration and washed with ethanol to ensure removal of any unbound drug. The complexes are usually air-dried in trays at room temperature.

Drug-resin complexes rapidly release the drug in the patient, such as, for example, in the gastrointestinal tract. For example, an Amberlite IR-120 phenylpropanolamine complex with a 35 percent drug loading released 61 percent of the drug in 60 minutes in a 0.1 N hydrochloric acid dissolution medium.

The amount of drug that can be loaded onto a resin will typically range from about 1% to about 50% by weight of the drug-resin particles. A skilled artisan with little or no experimentation can readily determine the optimum loading for any drug resin complex. In a preferred embodiment, loadings of about 5% to about 20% by weight of the drug-resin particles can be employed. For drugs such as dextramethoraphen and phenylpropanolamine, typical loadings of about 10% by weight of the drug-resin particles can be advantageously employed.

Impregnation

Drug-resin particles can be impregnated with a solvating agent basically as described in U.S. Pat. No. 4,221,778. The solvating agent can be added as an ingredient in the resin drug complexation step or preferably, the particles can be treated with the solvating agent after complexing. This treatment helps particles retain their geometry, and enables the effective application of diffusion barrier coatings to such particles. One preferred solvating agent is polyethylene glycol, a normally solid hydrophilic agent. Other effective solvating (impregnating) agents include, for example, propylene glycol, mannitol, lactose, methylcellulose, hydroxypropylmethylcellulose, sorbitol, polyvinylpyrrolidone, carboxypolymethylene, xanthan gum, propylene glycol alginate and combinations of these agents. The solvating agent may be present in an amount of up to about 30 parts by weight of the solvating agent to 100 parts by weight of the resin has been found to be effective. Preferably, the solvating agent is present in an amount of about 10 to about 25 parts by weight. Such pretreatment of drug-resin complex enables the effective application of diffusion barrier coatings, resulting in the ability to effectively prolong the release of drugs from drug resin complexes.

Diffusion Barrier Coating

Next, impregnated particles are coated with a diffusion barrier comprising a water-permeable, film-forming polymer. Any coating procedure which provides a contiguous coating on each particle of drug-resin complex without significant agglomeration of particles may be used. Coatings may be applied with a fluid-bed coating apparatus having the Wurster configuration. Measurements of particle size distribution can be done before and after coating to show that agglomeration of particles is insignificant.

The polymer may be any of a large number of natural or synthetic film-formers used singly, in admixture with each other, and in admixture with plasticizers, pigments and other substances to alter the characteristics of the coating. In general, the major components of the coating should be insoluble in and permeable to water. The water-soluble barrier comprise a pharmaceutically acceptable polymer such as, for example, ethylcellulose, methylcellulose, hydroxypropylmethylcellulose (HPMC), hydroxyethlycellulose (HEC), acrylic acid ester, cellulose acetate phthalate, HEC phthalate, HPMC phthalate or other cellulosic polymers, or mixtures of polymers. Additional examples of coating polymers are described by R. C. Rowe in Materials Used in Pharmaceutical Formulation (A. T. Florence, editor), Blackwell Scientific Publications, Oxford, 1-36 (1984), incorporated by reference herein. Preferably the diffusion barrier is ethyl cellulose, for example, an ethyl cellulose having the content of ethoxyl group from 44 to 47.5%, preferably from 45 to 46.5%. In embodiments of the present invention, the inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticizer the ethylcellulose before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such a dibutyl sebacate, diethyl phthalate, triethyl citrate, tributyl citrate and triacetin, although it is possible that other water-insoluble plasticizers (such as acetylated monoglycerides, phthalate esters, castor oil, etc.) may be used. A plasticizer such as Durkex 500 vegetable oil may also be incorporated to improve the film forming property. Preferably, it is desirable to incorporate a water-soluble substance, such as methyl cellulose, to alter the permeability of the coating.

One commercially available aqueous dispersion of ethylcellulose is Aquacoat® (FMC Corp., Philadelphia, Pa., U.S.A.). Aquacoat® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat® with a suitable plasticizer prior to use.

Another aqueous dispersion of ethylcellulose is commercially available as Surelease® (Colorcon, Inc., West Point, Pa., U.S.A.). This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly onto substrates.

The barrier coating materials are applied as an aqueous suspension. Optimum coat weight and coat thickness may be determined for each drug-resin complex and generally depend on the drug release characteristics of the resin for a particular drug. For example, for drug release times within about 1 hour to about 4 hours, the drug-resin complex may be coated with a light coat weight. A light coat weight is a coat weight present in the amount of about 10% to about 20% by weight of the dry resin. For drug release times from about 6 hours to 10 hours, a medium coat weight may be used, i.e. a coat weight present in the amount of 30% to about 35% by weight. For drug release times for about 12 hours, a heavy coat weight may be used, i.e. a coat weight of about 40% to 50% by weight of the dry resin. Typically, the water-permeable, film-forming polymer comprises from about 1% to about 60% by weight of the drug-resin complex, and preferably from about 20% to about 50% by weight of the dry resin. In terms of coat thickness, preferably, the diffusion barrier coat thickness is at least 10 microns and more preferably, the diffusion barrier coat thickness is from about 10 microns to about 50 microns.

Enteric Coating Compositions

Another embodiment of the present invention is directed to providing an enteric coating either on the drug-resin particle or on the barrier-coated resin-drug particles. As is known in the art, an enteric coating is intended to prevent the active ingredients in the preparation, or dosage form, from disintegrating in the stomach, and to allow the active ingredient(s) to be released once the dosage form has passed into the small intestinal tract. Thus, polymeric materials that are suitable for enteric coating applications should be insoluble in a low pH medium having typically having a value less than 3.5, but soluble in a higher pH medium typically having a value greater than 5.5. Thus, the objectives for using enteric coating materials in pharmaceutical dosage forms include (a) to protect the stomach from the harmful effect(s) of an active ingredient, (b) to protect the active ingredient from the adverse effect(s) of gastric fluid, (c) to deliver an active ingredient to a particular region of the intestine, and (d) to provide a sustained release dosage form to the gastrointestinal tract.

Polymers that are commonly used as enteric coatings in pharmaceutical preparations include cellulosic materials such as cellulose acetate phthalate (C-A-P), cellulose acetate trimellitate (C-A-T), cellulose acetate succinate (C-A-S), hydroxy-propyl methyl cellulose phthalate (HPMCP), hydroxypropyl methyl cellulose acetate succinate (HPMCAS) and carboxy methyl ethyl cellulose (CMEC). Other, non-cellulosic, polymers that are used as enteric coatings include copolymers of methacrylic acid and methyl methacrylate or ethyl acrylate, terpolymers of methacrylic acid, methacrylate, and ethyl acrylate, and polyvinyl acetate phthalate (PVAP).

The enteric coating is preferably applied to the barrier coated drug-resin complex, although in some embodiments it may be desirable to provide the enteric coating directly on the drug-resin complex or on a drug adsorbed on an inert substrate such as sugar spheres. The enteric coating can be present in amounts from about 1.5% to about 30% by weight based on the particle being coated. Preferably, the enteric coating is present in an amount from about 5% to about 15% by weight of the particle being coated.

Method of Manufacture

The drug-resin particles of the present invention can be manufactured using techniques and equipment commonly available in the art. For each step, the skilled artisan can easily determine the appropriate conditions for each resin or drug with little or no experimentation. Methods may have to be altered depending upon the type of resin, amount of coating, or type of drug, however, these alterations are well within the skill of the artisan.

Typically, the drug-resin complex or particle is made by dissolving the drug in a suitable amount of purified water followed by addition of the resin. After the mixture is mixed thoroughly, the water is decanted and the drug-resin complex is washed with purified water. If an impregnating or surfactant agent is to be added, after drying the drug-resin complex, a solution of the impregnating agent is added to the drug-resin complex, mixed thoroughly, and the mixture dried. Subsequently, the mixture is screened to remove any lumped material of undesired size. The screened mixture is then coated with an aqueous dispersion of diffusion barrier coating material using a Wurster coating system. The coating may be applied as a bottom spray or top spray. If necessary, the coated drug-resin complex may be screened to any desired size.

Optionally, after coating the coated drug-resin complex may be cured at a suitable temperature and for a suitable amount of time. Curing is intended to heat the coating polymer such that the polymer achieves a low energy configuration and lays flat over the surface to improve coating properties. Curing temperatures may be in the range of about 35° C. to about 100° C., preferably in the range of about 40° C. to about 60° C., and more preferably the curing temperature is in a range of about 45° C. to about 50° C. Curing times may be for about 2 hours to about 48 hours, preferably from about 4 hours to about 36 hours and more preferably, the curing time is from about 6 hours to about 24 hours.

Preparation of Pharmaceuticals

The coated drug-resin particles prepared according to the invention are suitable for preparing solid oral formulations using conventional materials and techniques. It is a preferred embodiment of the invention to suspend the coated drug-resin particles in an essentially aqueous vehicle with the only restrictions on its composition being (i) an absence of, or very low levels of ionic ingredients, and (ii) a limitation on the concentrations of water-miscible organic solvents, such as alcohol, to those levels which do not cause dissolution of the diffusion barrier coating.

Liquid forms such as syrups and suspensions preferably contain from about 1% to about 50% and more preferably from about 1% to about 25% and most preferably from about 3% to about 10% of the drug-resin complex. Liquid oral dosage forms include aqueous and nonaqueous solutions, emulsions, suspensions, and solutions and/or suspensions reconstituted from non-effervescent granules, containing suitable solvents, preservatives, emulsifying agents, suspending agents, diluents, sweeteners, coloring agents, and flavoring agents.

In preparing the liquid oral dosage forms, the coated drug-resin complexes are incorporated into an aqueous-based orally acceptable pharmaceutical carrier consistent with conventional pharmaceutical practices. An “aqueous-based orally acceptable pharmaceutical carrier” is one wherein the entire or predominant solvent content is water. Typical carriers include simple aqueous solutions, syrups, dispersions and suspensions, and aqueous based emulsions such as the oil-in-water type. Preferably, the carrier is a suspension of the pharmaceutical composition in an aqueous vehicle containing a suitable suspending agent. Suitable suspending agents include Avicel RC-591 (a microcrystalline cellulose/sodium carboxymethyl cellulose mixture available from FMC), guar gum and the like. Such suspending agents are well known to those skilled in the art. While the amount of water in the compositions of this invention can vary over quite a wide range depending upon the total weight and volume of the drug-resin complex and other optional non-active ingredients, the total water content, based on the weight of the final composition, will generally range from about 20 to about 75%, and, preferably, from about 20 to about 40%, by weight/volume.

Although water itself may make up the entire carrier, typical liquid formulations preferably contain a co-solvent, for example, propylene glycol, glycerin, sorbitol solution and the like, to assist solubilization and incorporation of water-insoluble ingredients, such as flavoring oils and the like into the composition. In general, therefore, the compositions of this invention preferably contain from about 5 to about 25 volume/volume percent and, most preferably, from about 10 to about 20 volume/volume percent, of the co-solvent.

As used herein, unless otherwise defined, the term “substantially free of organic solvent” means that the composition has less than 5% by weight of organic solvents, preferably, less than 2% by weight of the composition. More preferably, the term “substantially free of organic solvent” means that the composition has less than 1% by weight of organic solvents. Organic solvents include, but are not limited to, chloroform, methylene chloride, acetone, tetrahyrdrofuran, and the like.

The compositions of this invention may optionally contain one or more other known therapeutic agents, particularly those commonly utilized in cough/cold preparations, such as, for example, a decongestant such as pseudoephedrine hydrochloride, phenylpropanolamine HCl, phenylephrine hydrochloride and ephedrine hydrochloride; an analgesic such as acetaminophen and ibuprofen; an expectorant or mucolytic such as glyceryl guaiacolate, terpin hydrate, ammonium chloride, N-acetylcysteine and ambroxol; and an antihistamine such as chlorpheniramine maleate, doxylamine succinate, brompheniramine maleate and diphenhydramine hydrochloride: all of which are described in U.S. Pat. No. 4,619,934 to Sunshine et al., which is incorporated by reference herein. Also useful are bronchodilators such as theophylline and albuterol.

Other optional ingredients well known to the pharmacist's art may also be included in amounts generally known for these ingredients, for example, natural or artificial sweeteners, flavoring agents, colorants and the like to provide a palatable and pleasant looking final product, antioxidants, for example, butylated hydroxy anisole or butylated hydroxy toluene, and preservatives, for example, methyl or propyl paraben or sodium benzoate, to prolong and enhance shelf life.

Tailored Release Profiles

In accordance with another embodiment of the present invention, it is possible, by employing various combinations of free drug, drug-resin particles, barrier-coated drug-resin particles, enteric-coated drug resin particles, or barrier and enteric coated drug-resin particles described above, to tailor the release properties of a pharmaceutical preparation to provide a desired bioavailability profile. In this embodiment, the same or different drugs can be supplied in any of the following forms:

(1) free drug in solution;

(2) uncoated drug-resin complex;

(3) barrier coated drug-resin complex;

(4) enteric coated drug-resin complex;

(5) enteric coated, barrier coated drug-resin complex; and

(6) enteric coated free drug adsorbed on an inert substrate, e.g., sugar spheres.

One preferred combination approach according to the invention is the use of at least two different barrier coated drug-resin complexes, wherein the difference between the particles is the amount of barrier coating on each particle, so that the drug can be released at different rates from each type of barrier coated products. For example, a relatively light barrier coating on one portion of the total drug-resin complex mixed with a second portion coated with a relatively heavier barrier coating can result in the same or different drugs being release at two different rates.

In another preferred combination approach according to this invention is the use of barrier coated drug-resin complex with enteric coated barrier coated drug-resin complex. Systems with only barrier coated particles or barrier coated particles and free drugs are difficult to tailor for optimum release properties because these systems tend to quickly reach equilibrium conditions in the stomach. Applicant has discovered that these equilibrium effects can be overcome or delayed until after the complex leaves the stomach by employing the enteric coated or enteric coated particles described above. Such a system provides release profile not particularly achievable with the prior art approaches. Formulations of the present invention may release in vivo at least one drug over a period of about 4 hours, preferably over a period of 12 hours, and more preferably, the formulations of the present invention release in vivo at least one drug over a period of 24 hours.

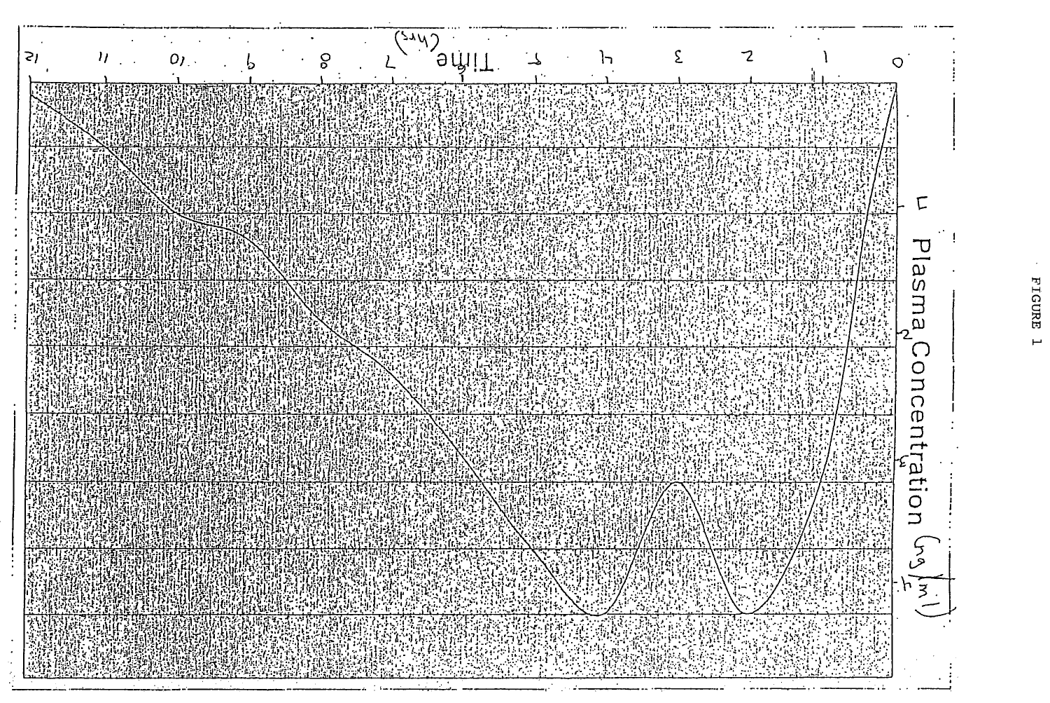

As a non-limiting example of such a tailored release approach, the system of the present invention can be employed to provide the effect of multiple doses of the drug as shown in FIG. 1. A serum profile (plasma concentration vs. time after administration) of this type can be achieved, for example, by providing barrier-coated drug-resin particles in combination with enteric coated particles (either barrier coated or uncoated drug-resin particles). FIG. 1 illustrates the profile of a pharmaceutical formulation comprising a mixture of barrier coated methylphenidate and enteric coated methylphenidate. The barrier coated drug is a lightly barrier coated drug, i.e. the barrier coating is about 20% by weight of the coating to the uncoated resin. A 15 mg dose is administered, and over a 12 hour period, the drug releases and provides two plasma concentration peaks. The first peak has a Cₘₐₓ of 4.2±1 ng/ml at two hours, the second peak has a Cₘₐₓ of 4.2+1 ng/ml at 4 hours. Thereafter, the drug plasma concentration gradually decreases over time.

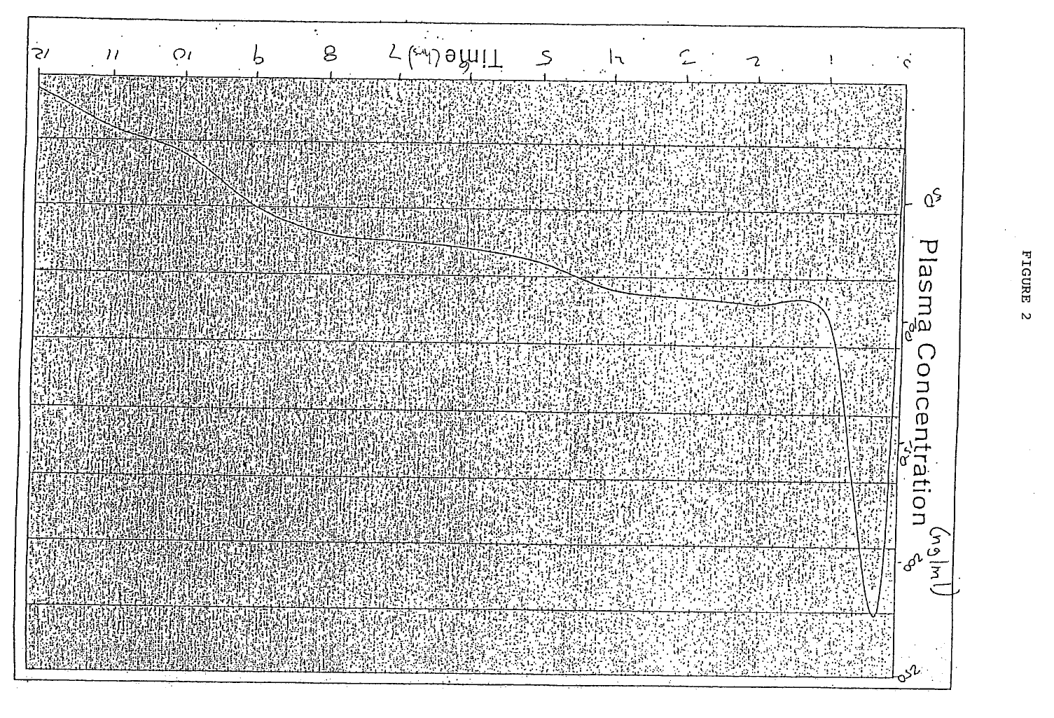

FIG. 2 shows another serum profile that can be tailored according to the present invention. This type of profile, which includes immediate high-level release and extended release characteristics, can be prepared, for example, by combining free drug, barrier coated drug-resin complex and enteric coated barrier coated drug-resin complex. The enteric coated part of this formulation prevents solution equilibrium effects from eliminating the extended release of the drug, as might be the case with only free drug and barrier coated drug. FIG. 2 illustrates the plasma concentration of pseudo-ephedrine, wherein the composition comprises free drug and a barrier coated drug. The barrier coated drug is a medium coated drug, i.e. the barrier coating is about 40% by weight of the coating to uncoated resin. A 120 mg dose is administered and over a 12 hour period, the free drug releases and provides an immediate peak in drug plasma concentration of Cₘₐₓ of about 230 ng/ml within 30 minutes, thereafter, the drug plasma concentration slowly drops off to about 50% to 20% of the Cₘₐₓ of 230 ng/ml for an additional 10 hours.

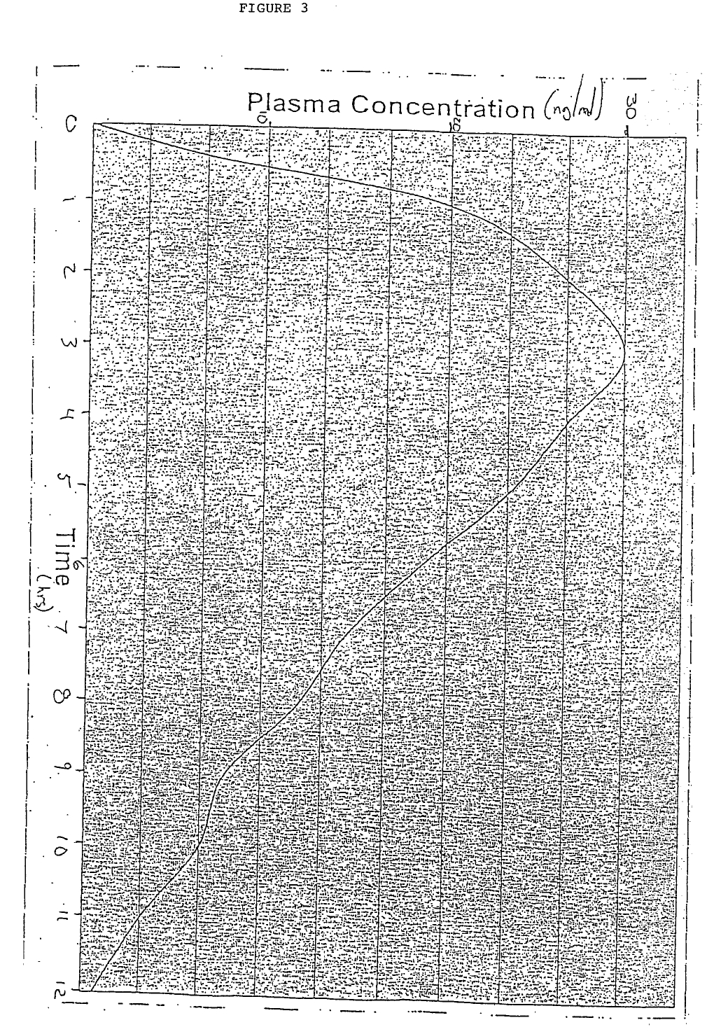

FIG. 3 shows another serum profile that can be tailored according to the present invention. This type of profile can be prepared, for example, by using just barrier coated drug-resin complex. FIG. 3 illustrates the drug plasma concentration of alprazolam, wherein the drug forms a drug-resin complex with a 30% by weight diffusion barrier coating. A 2 mg dose is administered and over a 12 hour period, the drug plasma concentration peaks at a Cₘₐₓ of about 30 ng/ml in about 3 hours followed by a slow drop-off over nine hours.

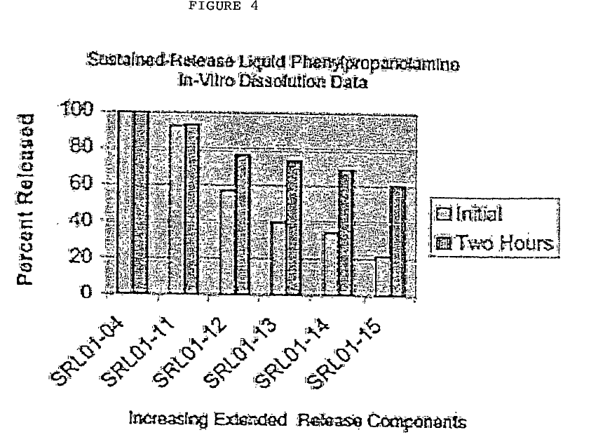

FIG. 4 illustrates the drug serum profile of PPA at time zero and after two hours. The Formulations 1-6 of the invention are described below. Formulation 1 released PPA immediately, such that at time zero the concentration of PPA equal 100%. At time zero, the amount of PPA released was as follows: Formulation 2 (95%), Formulation 3 (58%), Formulation 4 (40%), Formulation 5 (32%), and Formulation 6 (22%). After two hours, the amount of PPA released was Formulation 1 (100%), Formulation 2 (96%), Formulation 3 (78%), Formulation 4 (74%), Formulation 5 (70%), and Formulation 6 (60%).

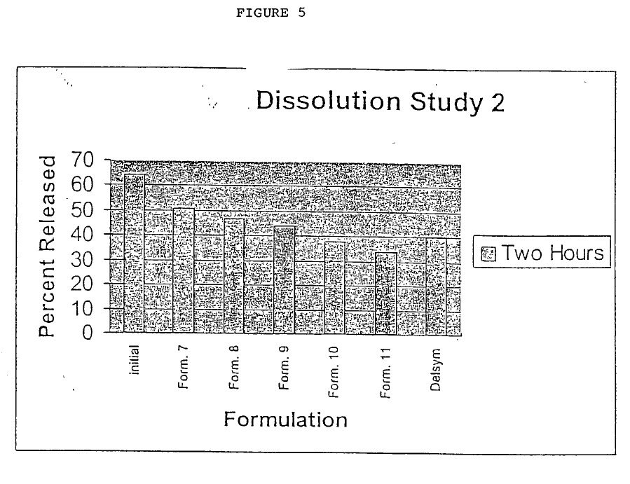

FIG. 5 illustrates the percent drug released of untreated drug-resin particles, formulations 7, 8, 9, 10, and 11 of the invention, and commercially available Delsym™ over a two hour period. The untreated composition released dextromethorphan the most quickly, while formulations 7, 8, and 9 released dextromethorphan more slowly than the untreated composition, but quicker than Delsym™. Delsym™, however, released dextromethorphan more quickly than Formulations 10 and 11. FIG. 5 illustrates the versatility of the methods of the present invention to tailor a formulation to release a drug at various rates.

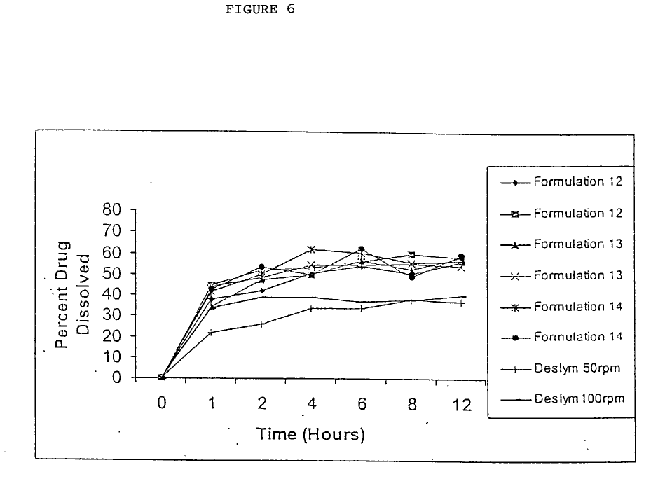

In FIG. 6, three formulations of the invention were compared to commercially available Delsym™ over a 12 hour period. Each formulation was placed in 0.1 N HCl USP Apparatus II stirred at 50 or 100 rpm. At time zero, and after one, two, four, six, eight, and 12 hours, a sample was taken to determine the amount of dextromethorphan present as a percent amount released over the total amount of dextromethorphan present in the formulation. Formulation 12 has 40% by weight of barrier coating material (applied by bottom spraying), Formulations 13 and 14 have 30% by weight of barrier coating applied by bottom spraying or top spraying, respectively. All formulations of the invention released a greater amount of dextromethorphan release compared to Delsym™.

Other embodiments and uses of the invention will be apparent to those skilled in the art from consideration of the specification and practice of the invention disclosed herein. All references cited herein for whatever reason, including all U.S. and foreign patents and patent applications, are specifically and entirely incorporated by reference. It is intended that the specification be considered exemplary only.

### EXAMPLES

The invention is further defined by reference to the following examples describing in detail, the preparation of the formulations, and the administration of the formulations of the present invention. It will be apparent to those skilled in the art, that many modifications, both to materials, and methods, may be practiced without departing from the purpose and interest of this invention. Accordingly, the following examples are intended to be illustrative of the present invention and should not be construed, in any way, to be a limitation thereof.

### Example 1

### Preparation of Phenylpropanolamine Formulations

Generally the formulations of the invention were prepared using standards techniques and equipment. Using a mixer the drug-resin complex was made by dissolving the drug, phenylpropanolamine (PPA), in purified water and thereafter, adding the polystyrene. The mixture was stirred thoroughly. Thereafter, the water was decanted and the drug-resin complex was washed with purified water. Using a fluid bed dryer, a surfactant agent, PEG, was added to the mixture, mixed, and the mixture dried. The dried drug-resin complex was screened for size to avoid lumps, and later coated with an aqueous dispersion of ethylcellulose using a Wurster coating system (Glatt Wurster Coater). Thereafter, the barrier coated drug-resin complex was milled as needed and passed through a screen to remove agglomerates. In total six formulations of PPA-resin complex were prepared the amount of coating is given in parenthesis as a weight percent of coating/dry resin weight. The barrier coating material for formulations 2-6 was Opadry® (Colorcon, West Point, Pa., 19486-0024), however, formulations 3-6 were additionally coated with a second barrier coating material, Surelease®. Formulation 1 was the control uncoated PPA and Formulation 2 was coated with Opadry® only. Formulations 3-6 were coated with different amounts of barrier coating, which is given as a weight percentage in parenthesis, to provide Formulation 3 (10%), Formulation 4 (15%), Formulation 5 (20%), and Formulation 6 (25%).

### Example 2

### Preparation of Dextromethorphan Formulations

Using the methodology outline in Example 1, five dextromethorphan formulations were made. In each formulation, the amount of Surelease® coating by weight percent of dry uncoated resin is given in parenthesis. The formulations prepared were Formulation 7 (19%), Formulation 8 (24%), Formulation 9 (29%), Formulation 10 (39%), and Formulation 11 (49%).

### Example 3

### Dissolution Study of PPA

The release profile of PPA was studied using the formulations of Example 1. Each formulation was dissolved in 0.1 N HCl solution using an USP Apparatus II while stirring using mixing paddles set at 100 rpm. At each time interval, a sample of the solution was analyzed to determine the presence and amount of PPA. Two datapoints were taken one at time zero (initial) and a second at a time of two hours. Formulation 1 (SRL01-04) released PPA immediately, such that at time zero the concentration of PPA equal 100%. At time zero, the amount of PPA released was as follows: Formulation 2 (95%) (SRL01-11), Formulation 3 (58%) (SRL01-12), Formulation 4 (40%) (SRL01-13), Formulation 5 (32%) (SRL01-14), and Formulation 6 (22%) (SRL01-15). After two hours, the amount of PPA released was Formulation 1 (100%), Formulation 2 (96%), Formulation 3 (78%), Formulation 4 (74%), Formulation 5 (70%), and Formulation 6 (60%). The time the coated formulations released PPA correlated to amount of drug coating, i.e. the higher the percent of drug, the less amount of drug released. FIG. 4 summarizes this data in graphic form.

### Example 4

### Dissolution Study 1 of Dextromethorphan

Formulation 9 and Formulation 10 from Example 2 where compared against commercially available DelSym™. The release profile of dextromethorphan was studied over a 12 hour period. Each formulation was dissolved in 0.1 N HCl solution using an USP Apparatus II while stirring using mixing paddles set at 100 rpm. At each time interval, a sample of the solution was analyzed an appropriate Multi-Cell UV/VIS spectrophotometer meter to determine the presence and amount of dextromethorphan. The generally accepted method for demonstrating equivalency of dissolution curves uses the logarithmic reciprocal square root transformation of the sum of squared error defined as the similarity factor “f₂,” which is given by the formula:

f₂=50*log{[1+(1/n)Σ(Rₜ−Tₜ)²]^{0.5}*100]

in FDA Guidance Documents. See, Dissolution Testing of Immediate Release Solid Oral Dosage Forms, Guidance for Industry, U.S. Food and Drug Administration, August 1997. The FDA accepts a f₂ value of greater than 50 as demonstration of equivalent dissolution curves. Table 1 summarizes the comparative dissolution data.

TABLE 1
f₂ value calculation for formulations 9, 10, and Delsym ™

| Time (hrs) | Formulation 9 | Formulation 10 | Delsym ™ |
|---|---|---|---|
| 1 | 47 | 39 | 41 |
| 2 | 51 | 45 | 44 |
| 4 | 53 | 47 | 45 |
| 6 | 54 | 48 | 46 |
| 8 | 54 | 48 | 46 |
| 12 | 54 | 48 | 48 |
| f₂ vs. Delsym ™ | 57 | 85 | 100 |

Both Formulation 9 and Formulation 10 were considered equivalent or better than Delsym™ as the f₂ values exceed 50, i.e., 57 and 85, respectively. Consequently, Formulations 1 and 2 demonstrated the ability of the present invention to create multiple formulations capable of releasing a drug of interest over several time periods depending on need.

### Example 5

### Dissolution Study 2 of Dextromethorphan

The five formulations of Example 2 were compared to untreated resin-drug complex and commercially available DelSym™ over a two hour period. Each formulation was places in 0.1 N HCl USP Apparatus II stirred at 100 rpm. After two hours, a sample was taken to determine the amount of dextromethorphan present as a percent amount released over the total amount of dextromethorphan present in the formulation. All formulations of the invention delayed dextromethorphan release compared to untreated drug-resin complex. Formulation 7 (51%), Formulation 8 (47%), and Formulation 9 (44%) released more dextromethorphan than Delsym™ (40%), however, Formulation 10 (38%) and Formulation 11 (34%) released less dextromethorphan than Delsym™ over the two hour period. Accordingly, Dissolution Study 2 demonstrated that the formulations of the present inventions can be formulated to selectively release a specific amount of drug. FIG. 5 summarizes the comparative data.

### Example 6

### Dissolution Study 3 of Dextromethorphan

Using the method of Example 1, three formulations of dextromethorphan were prepared. The three formulations were compared to commercially available Delsym™ over a 12 hour period. Each formulation was placed in 0.1 N HCl USP Apparatus II stirred at 50 or 100 rpm. At time zero, and after one, two, four, six, eight, and 12 hours, a sample was taken to determine the amount of dextromethorphan present as a percent amount released over the total amount of dextromethorphan present in the formulation. All formulations of the invention released a greater amount of dextromethorphan release compared to Delsym™. Formulation 12 has 40% by weight of barrier coating material (applied by bottom spraying), Formulations 13 and 14 have 30% by weight of barrier coating applied by bottom spraying or top spraying, respectively. Table 2 summarizes time, the percent by weight of the dissolved dextromethorphan, and the paddle speed. FIG. 6 illustrates in graphical form the data.

TABLE 2
Dissolution Comparison of Formulations 12, 13, 14, and Delsym ™

| | Weight Percent of Dissolved Dextromethorphan at | | | | | | | |
|---|---|---|---|---|---|---|---|---|
| | Time (hours) | | | | | | | |
| Form. | 0 | 1 | 2 | 4 | 6 | 8 | 12 | RPM |
| 12 | 0 | 38.11 | 42.13 | 50.56 | 54.01 | 51.41 | 55.91 | 50 |
| 12 | 0 | 44.55 | 52.09 | 53.05 | 56.21 | 59.54 | 57.93 | 100 |
| 13 | 0 | 34.51 | 47.15 | 49.91 | 56.61 | 52.71 | 58.02 | 50 |
| 13 | 0 | 43.92 | 48.15 | 54.59 | 54.47 | 54.82 | 54.00 | 100 |
| 14 | 0 | 41.70 | 49.90 | 61.92 | 60.11 | 55.76 | 56.68 | 50 |
| 14 | 0 | 42.68 | 53.47 | 49.96 | 62.34 | 48.73 | 59.20 | 100 |
| Delsym | 0 | 21.96 | 25.77 | 33.59 | 33.81 | 37.83 | 36.89 | 50 |
| Delsym | 0 | 33.51 | 38.95 | 38.72 | 36.72 | 38.13 | 39.94 | 100 |

### Example 7

### In vivo Study of a Methylphenidate Formulation

A methylphenidate composition is prepared using the methodology of Example 1 to prepare two differently coated drug-resin complexes. One drug-resin complex has only a light barrier coating weight, i.e. a particle coated having about 20% by weight of the resin. The second drug-resin complex has an enteric coating in addition to the light barrier coating weight. Thereafter, the particles are mixed into one liquid composition. The composition is administered to a human in a 15 mg dose and the serum profile of the methylphenidate formulation is monitored. FIG. 1 illustrates the serum profile of a pharmaceutical formulation comprising a mixture of barrier coated methylphenidate and the same particles further coated with an enteric coating. Over a 12 hour period the drug release characteristics provided two plasma concentration peaks. The first peak and second peaks are at concentrations of about 4.2 ng/ml at two and four hours, respectively. Thereafter, the drug serum concentration gradually decreases over time.

### Example 8

### In vivo study of a Pseudoephedrine Formulation

A pseudoephedrine composition is prepared using the methodology of Example 1 to prepare a coated drug-resin complex. A medium barrier coated drug-resin complex, i.e. the barrier coating is about 40% by weight of the coating to uncoated drug-resin complex is prepared. Thereafter, the free drug and drug-resin complex are mixed into a liquid composition. The composition is administered to a human in a 120 mg dose and the serum profile of the pseudoephedrine formulation is monitored. FIG. 2 illustrates the drug plasma concentration profile for pseudoephedrine. Over a 12 hour period, the free drug provides an immediate peak in drug plasma concentration of a Cₘₐₓ of 230 ng/ml within 30 minutes, thereafter, the drug serum concentration slowly drops off to about 50% to 20% of the maximum concentration for an additional 10 hours.

### Example 9

### In vivo Study of Alprazolam

An alprazolam composition is prepared using the methodology of Example 1 to prepare a coated drug-resin complex. A medium barrier coated drug-resin complex, i.e. the barrier coating is about 30% by weight of the coating to uncoated drug-resin complex is prepared. Thereafter, the drug-resin complex is mixed into a liquid composition. The composition is administered as a 2 mg dose to a human and the serum profile of the alprazolam formulation is monitored. FIG. 3 illustrates the serum profile. Over a 12 hour period, the drug plasma concentration slowly peaks to a Cₘₐₓ of 30 ng/ml in about 3 hours followed by a slow drop-off over nine hours.

What is claimed is:

1. An oral pharmaceutical composition comprising ion-exchange resin particles having particle sizes from 30 microns to about 500 microns; at least one pharmacologically active drug releasably bound to the particles to form drug-resin complexes, wherein the drug-resin complexes are coated with an aqueous based diffusion barrier which comprises from about 1 percent to about 60 percent, by weight of the resin particles, of a water-permeable, film-forming polymer.

2. The composition of claim 1 wherein the particle size is from about 40 microns to about 150 microns.

3. The composition of to claim 1 wherein the particles are regularly shaped, irregularly shaped, or both.

4. The composition of claim 1 wherein the resin has an ion-exchange capacity of less than 6.0 meq./g.

5. The composition of claim 1 wherein the drug comprises from about 1 percent to about 50 percent by weight of the drug-resin particles.

6. The composition of claim 1 wherein the water-permeable polymer comprises ethyl cellulose.

7. The composition of claim 1 wherein the water-permeable, film-forming polymer contains no substantial traces of an organic solvent.

8. The composition of claim 1 which provides a controlled release of active drug in vivo.

9. The composition of claim 1 wherein the particles contain an impregnating agent.

10. The composition of claim 9 wherein the impregnating agent comprises polyethylene glycol.

11. The composition of claim 9 wherein the impregnating agent comprises a methacrylic acid polymer.

12. The composition of claim 1 wherein the pharmacologically-active drug is selected from the group consisting of antitussive expectorants, bronchodilators, antihistamines, digestive tract antispasmodics, antipsychotic drugs, antianxiety drugs, antidepressants, antipyretic analgesics, opioid analgesic drugs, coronary dilators, hypotensive drugs, peripheral vasodilators/vasoconstrictors, antibiotics, chemo-therapeutic drugs, antituberculosis drugs, and antiprotozoan drugs.

13. The composition of claim 1 wherein the pharmacologically-active drug is selected from the group consisting of dehydrocholic acid, diflunisal, ethacrynic acid, fenoprofen, furosemide, gemfibrozil, ibuprofen, naproxen, phenytoin, probenecid, sulindac, theophylline, salicylic acid, acetylsalicylic acid, acetophenazine, amitriptyline, amphetamine, benztropine, biperiden, bromodiphenhydramine, brompheniramine, carbinoxamine, chlorcyclizine, chlorpheniramine, chlorphenoxamine, chlorpromazine, clemastine, clomiphene, clonidine, codeine, cyclizine, cyclobenzaprine, cyproheptadine, desipramine, dexbrompheniramine, dexchlorpheniramine, dextroamphetamine, dextromethorphan, diazepam, dicyclomine, diphemanil, diphenhydramine, doxepin, doxylamine, ergotamine, fexofenadine, fluphenazine, haloperidol, hydrocodone, hydroxychloroquine, hydroxyzine, hyoscyamine, imipramine, levopropoxyphene, maprotiline, meclizine, mepenzolate, meperidine, mephentermine, mesoridazine, methadone, methdilazine, methscopolamine, methysergide, metoprolol, nortriptylene, noscapine, nylindrin, orphenadrine, papaverine, pentazocine, phendimetrazine, phentermine, phenylpropanolamine, pyrilamine, tripelennamine, triprolidine, promazine, propoxyphene, propanolol, pseudoephedrine, pyrilamine, quinidine, scopolamine, dextromethorphan, chlorpheniramine, aminocaproic acid, aminosalicylic acid, hydromorphone, isoxsuprine, levorphanol, melphalan, morphine, nalidixic acid, and paraaminosalicylic acid.

14. The composition of claim 1 wherein at least some of the diffusion barrier coated particles are coated with an enteric coating.

15. The composition of claim 1 wherein the composition is a liquid composition.

16. A method for manufacturing coated particles for use in the manufacture of a prolonged release preparation comprising:
contacting particles of an ion-exchange resin with a pharmaceutically active drug to form a drug-resin complex wherein the particle size is from about 30 microns to about 500 microns; and
coating the drug-resin complex with an aqueous suspension of a water-permeable, film-forming polymer such that the resulting coatings have an average thickness of at least about 10 microns.

17. The method of claim 16 wherein the particle size is from about 40 microns to about 150 microns.

18. The method of claim 16 wherein the drug comprises from about 1 percent to about 50 percent by weight of the drug-resin complex.

19. The method of claim 16 wherein the water-permeable, film-forming polymer comprises ethyl cellulose.

20. The method of claim 16 wherein the water-permeable, film-forming polymer contains no substantial traces of an organic solvent.

21. The method of claim 16 further comprising applying an impregnating agent to the particles.

22. The method of claim 21 wherein the impregnating agent is polyethylene glycol.

23. The method of claim 21 wherein the impregnating agent is a methacrylic acid polymer.

24. A pharmaceutical composition comprising:
ion-exchange resin particles having particle sizes from about 30 microns to about 500 microns;
at least one pharmacologically active drug releasably bound to the particles to form drug-resin complexes; and
a pharmaceutically acceptable carrier, wherein the drug-resin complexes are coated with an aqueous based diffusion barrier which comprises from about 1 percent to about 60 percent, by weight of the resin particles, of a water-permeable, film-forming polymer.

25. The composition of claim 24 wherein the pharmaceutically acceptable carrier is a liquid.

26. The composition of claim 24 further comprising from about 1.5 percent to about 30 percent by weight of enteric coated barrier-coated drug-resin complex particles.

27. The composition of claim 24 further comprising free drug that is not bound to resin.

28. The pharmaceutical composition according to claim 24, wherein the drug-resin complexes comprise at least a first portion having a first diffusion barrier coating weight and a second portion having a different diffusion barrier coating weight.

29. The pharmaceutical composition according to claim 24, wherein the composition is a liquid.

30. A method for the controlled administration of a drug comprising:
administering to a patient a therapeutically acceptable dose of a composition comprising a diffusion barrier coated drug-resin particle wherein the diffusion barrier is present in an amount of about 1 percent to about 60 percent by weight of the drug-resin particles and the diffusion barrier is a water-permeable, film-forming polymer.

31. The method of claim 30 wherein the diffusion barrier comprises ethyl cellulose.

32. The method of claim 30 wherein the diffusion barrier contains no substantial traces of an organic solvent.

33. The method of claim 30 further comprising applying an impregnating agent to the drug-resin complexes.

34. The method of claim 33 wherein the impregnating agent is polyethylene glycol.

35. The method of claim 33 wherein the impregnating agent is a methacrylic acid polymer.

36. The method of claim 30 wherein the composition further comprises drug that is not bound to resin.

37. The method of claim 30 wherein the drug is released in vivo over a period of about 4 hours.

38. The method of claim 30 wherein the drug is released in vivo over a period of about 12 hours.

39. The method of claim 30 wherein the drug is released in vivo over a period of 24 hours.

40. The method of claim 30 wherein the drug-resin particles are from about 30 microns to about 500 microns in size.

41. The method of claim 30 wherein the drug-resin particles are from about 40 microns to about 150 microns in size.

42. The method of claim 30, wherein the composition is a liquid.

43. A method of formulating a drug product with a tailored serum profile comprising the step of combining at least two drug forms selected from the group consisting of free drug, free drug complexed with an ion exchange resin, free drug adsorbed on an inert substrate, barrier coated ion exchange resin-drug complex, barrier coated adsorbed drug on an inert substrate, enteric coated adsorbent drug on an inert substrate, enteric coated ion exchange resin-drug complex, and enteric coated barrier coated ion exchange resin-drug complex.

44. The method according to claim 43 further comprising the step of dispersing the drug forms in a pharmaceutically acceptable carrier.

45. The method according to claim 44, wherein the carrier is a liquid.

46. The method according to claim 43, wherein the drug forms are barrier coated ion exchange resin-drug complex and enteric coated barrier coated ion exchange resin-drug complex.

47. The method according to claim 43, wherein the drug forms are barrier coated ion exchange resin-drug complex and free drug.
